(19) United States
(12) Patent Application Publication
(10) Pub. No.: US 2021/0100540 A1
(43) Pub. Date: Apr. 8, 2021

(54) ROLLABLE TISSUE SPECIMEN BAG

(71) Applicant: COVIDIEN LP, Mansfield, MA (US)

(72) Inventors: JACOB C. BARIL, NORWALK, CT (US); SAUMYA BANERJEE, HAMDEN, CT (US); GEORGE S. MATTA, PLAINVILLE, MA (US); AMY L. KUNG, HAMDEN, CT (US); CHRISTOPHER M. MEEHAN, NORTH HAVEN, CT (US); JASON T. MICKUS, AVON, CT (US); SCOTT J. PRIOR, SHELTON, CT (US); THOMAS A. ZAMMATARO, HAMDEN, CT (US)

(21) Appl. No.: 16/595,119

(22) Filed: Oct. 7, 2019

(51) Int. Cl. A61B 17/00

(52) U.S. Cl. CPC A61B 17/00234; A61B 2017/00867; A61B 2017/00862; A61B 2017/00287

(57) ABSTRACT

A tissue specimen retrieval bag assembly includes a tissue specimen bag having an open proximal end and a closed distal end, the proximal end including a cuff defined therein and extending therearound. A flexible bag brim is disposed within the cuff and is transitionable between a first, collapsed configuration and a second, expanded configuration. The flexible bag brim includes a cross section configured to facilitate both furling the tissue specimen bag onto itself around the bag brim when the bag brim is disposed in the second, expanded configuration and securing the tissue specimen bag in a desired furled configuration.

### BACKGROUND

### Technical Field

The present disclosure relates to tissue specimen retrieval from an internal body cavity and, more particularly, to specimen retrieval or specimen containment bags that facilitate retrieval of a tissue specimen from an internal body cavity.

### Background of Related Art

In minimally-invasive surgical procedures, operations are carried out within an internal body cavity through small entrance openings in the body. The entrance openings may be natural passageways of the body or may be surgically created, for example, by making a small incision into which an access device is inserted.

Minimally-invasive surgical procedures may be used for partial or total retrieval of a tissue specimen from an internal body cavity. However, the restricted access provided by minimally-invasive openings (natural passageways and/or surgically created openings) presents challenges with respect to maneuverability and visualization. The restricted access also presents challenges when the tissue specimen is required to be removed. As such, a tissue specimen that is deemed too large for intact retrieval may be broken down into a plurality of smaller pieces to facilitate retrieval from the internal body cavity.

During such minimally-invasive surgical procedures, it is common that a cyst, tumor, or other affected tissue specimen is required to be removed. In these and other procedures where cancerous tissue is required to be removed, retrieval of the tissue specimen in an enclosed environment such as a specimen retrieval bag or containment bag is highly desirable to inhibit seeding of cancer cells. Thus, with respect to breaking down large tissue specimens for retrieval through minimally-invasive openings, there is the added challenge of doing so within an enclosed environment.

In these instances, a standalone tissue bag may be utilized to contain large tissue specimens such as a uterus for hysterectomies or fibroids for myomectomies. The specimen retrieval bag or containment bag typically includes a bag brim having a flexible wire support that is transitionable between a first collapsed configuration for insertion through an incision or natural body orifice and a second expanded configuration for encapsulating tissue specimens. The bag brim, once externalized, may be manipulated or rolled to enhance surgical access to the tissue specimen or “tent” the specimen as needed.

### SUMMARY

As used herein, the term “distal” refers to the portion that is described which is further from a user, while the term “proximal” refers to the portion that is being described which is closer to a user. The terms “substantially” and “approximately,” as utilized herein, account for industry-accepted material, manufacturing, measurement, use, and/or environmental tolerances. Further, any or all of the aspects and features described herein, to the extent consistent, may be used in conjunction with any or all of the other aspects and features described herein.

In accordance with aspects of the present disclosure is a tissue specimen retrieval bag assembly that includes a tissue specimen bag having an open proximal end and a closed distal end, the proximal end including a cuff defined therein and extending therearound. A flexible bag brim is included and is disposed within the cuff and is transitionable between a first, collapsed configuration and a second, expanded configuration. The flexible bag brim includes a cross section configured to facilitate both furling the tissue specimen bag onto itself around the bag brim when the bag brim is disposed in the second, expanded configuration and securing the tissue specimen bag in a desired furled configuration.

In aspects according to the present disclosure, the cross section includes a D-shaped cross section. In other aspects according to the present disclosure, the cross section includes a triangular cross section. The triangular cross section may include three sides, each of the three sides covered with a high friction material to facilitate gripping the tissue specimen bag when furling.

In still other aspects according to the present disclosure, the cross section includes a trilobular-shaped cross section. The trilobular-shaped cross section may include three edges defining three concave sections therebetween, the edges are configured to facilitate gripping the tissue specimen bag when furling.

In other aspects according to the present disclosure, the cross section includes a D-shaped cross section including a flat side and an arcuate side, one or both of the sides including a high friction material to facilitate gripping the tissue specimen bag when furling.

In still other aspects according to the present disclosure, the tissue specimen bag is made from nylon or polyurethane.

In accordance with other aspects of the present disclosure is a tissue specimen retrieval bag assembly that includes a tissue specimen bag having an open proximal end and a closed distal end, the proximal end including a cuff defined therein and extending therearound. A flexible bag brim is disposed within the cuff and includes a pair of flexible wires separated by one or more connectors. The pair of wires is transitionable between a first, collapsed configuration and a second, expanded, loop-like configuration. The connector(s) is configured to space the wires relative to one another along the length of the bag brim. The pair of wires and the connector(s) are configured to facilitate both furling the tissue specimen bag onto itself around the bag brim when the bag brim is disposed in the second, expanded, loop-like configuration and securing the tissue specimen bag in a desired furled configuration.

In other aspects according to the present disclosure, the pair of wires is made from a shape memory alloy. In yet other aspects according to the present disclosure, the shape memory alloy includes a nickel-titanium alloy or a copper-aluminum-nickel.

### BRIEF DESCRIPTION OF THE DRAWINGS

The above and other aspects and features of the present disclosure will become more apparent in view of the following detailed description when taken in conjunction with the accompanying drawings wherein like reference numerals identify similar or identical elements.

FIG. 1 is a perspective view of a tissue specimen bag provided in accordance with aspects of the present disclosure;

FIG. 2A is a cross sectional view of the area of detail of FIG. 1 showing one embodiment of a trilobular, rollable bag brim design;

FIG. 2B is a cross sectional view of the area of detail of FIG. 1 showing another embodiment of a trilobular, rollable bag brim design;

FIG. 3A is a perspective view of another embodiment of a tissue specimen retrieval bag having a flexible wire-like bag brim according to the present disclosure;

FIG. 3B is an enlarged view of the area of detail of FIG. 3A;

FIG. 3C is an enlarged view of the wire-like bag brim of FIG. 3A detached from the tissue specimen retrieval bag;

FIG. 4A is a perspective view of another embodiment of a tissue specimen retrieval bag having a rollable bag brim according to the present disclosure;

FIG. 4B is an enlarged, cross sectional view of the rollable bag brim of FIG. 4A; and

FIGS. 5A-5C are various views of the tissue specimen retrieval bag of FIGS. 4A and 4B for use with a tissue specimen “T”.

### DETAILED DESCRIPTION

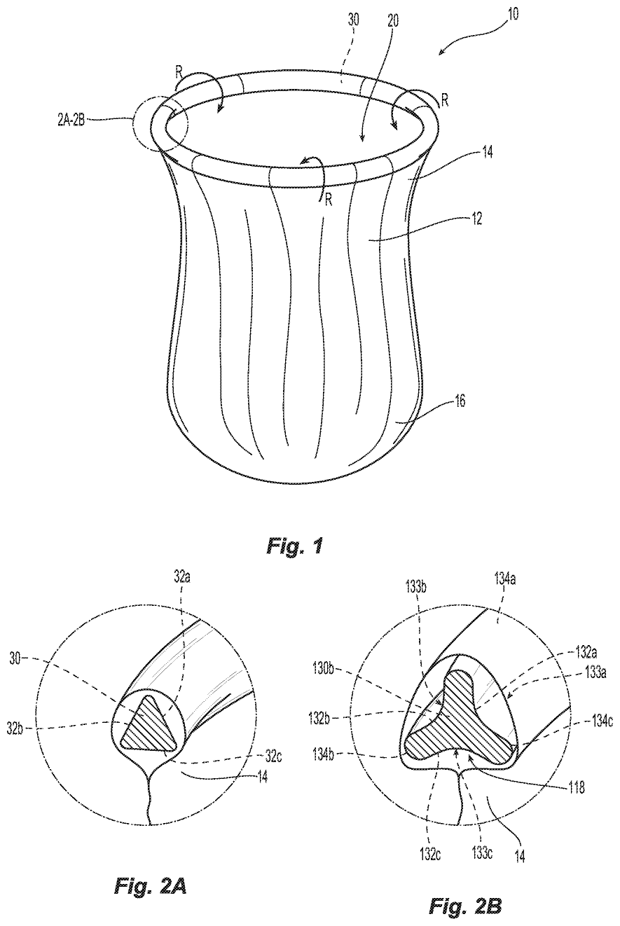

Turning initially to FIGS. 1-2A, one embodiment of a standalone tissue specimen retrieval bag or tissue containment bag assembly is shown and is represent by reference numeral 10. Bag assembly 10 includes a bag 12 having a proximal end 14 including an opening 20 defined therein and an enclosed distal end 16. Bag assembly 10 includes bag rim 30 that is configured to support bag 12 in such a fashion as to define opening 20 when the bag 12 is unfurled or in an open configuration. Opening 20 is of sufficient dimension to receive one or more tissue specimens “T” during a particular surgical procedure.

Bag rim 30 is configured to be flexible such that the bag brim 30 is easily transitionable between a first, collapsed configuration wherein the bag 12 is furled (as explained below) and a second, expanded configuration which allows the bag 12 to be unfurled for receipt of a tissue specimen “T”. As such, bag brim 30 may be made from any flexible material that is easily expandable from a collapsed configuration. Bag 12 may include any suitable rollable material such as nylon, polyurethane, etc.

Turning now to FIG. 2A, the bag brim 30 is configured to seat within an elongated cuff 18 defined in the proximal end of the bag 12. More particularly, bag brim 30 is of sufficient dimension to fit within the cuff 18 along an entire length thereof. Bag brim 30 may include two mating ends that, when engaged, form a generally circular configuration when opened to support bag 12 thereon. Bag brim 30 is generally triangular in shape and includes three sides 32a, 32b and 32c. Other geometric configurations or multi-sided arrangements are also envisioned and may be tailored for a particular purpose.

The generally triangular shape of the bag brim 30 allows the bag 12 to roll or furl around itself which, in turn, allows a surgeon to tent (i.e., position) the tissue specimen “T” closer to the proximal end of the operating cavity as needed for dissection. More particularly, the geometry of the sides 32a, 32b and 32c of the bag brim 30 facilitate furling/unfurling the bag 12 as needed and securing the bag 12 in a desired furled position. Bag 12 may be furled either inwardly or outwardly depending upon a particular purpose.

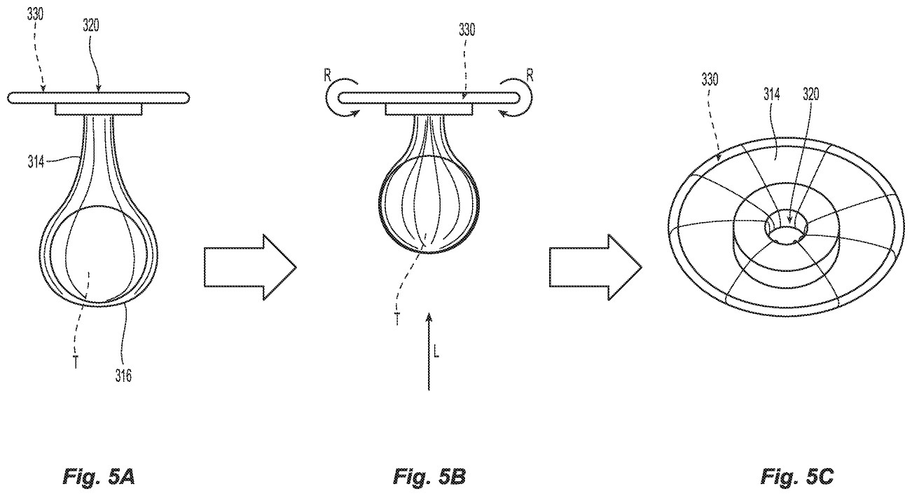

In use, the specimen “T” is placed into the specimen bag 12 through opening 20. The weight of the specimen “T” causes the specimen “T” to fall to toward the distal end 16 of the bag 12. The proximal end 14 of the bag 12 and the circularly-shaped bag brim 30 maintain the proximal end 14 the bag 12 outside the operating cavity (See for example, FIG. 5C). If the surgeon desires to bring the specimen “T” closer to the proximal end 14 of the bag 12, the surgeon furls the bag 12 around the bag brim 30 in the direction “R”. The triangular shape of the bag brim 30 facilitates furling the bag 12 and the brim 30 over on itself which, in turn, allows the surgeon to position the tissue specimen “T” at a desired depth “L” (See for example, FIG. 5B) within the surgical cavity depending upon a particular purpose. The sides 32a, 32b and 32c of the bag brim 30 may include a high friction surface to facilitate gripping the bag 12 when furling.

FIG. 2B shows an alternative triangular-shaped bag brim 130 disposed within a cuff 118 having three generally rounded sides 132a, 132b and 132c separated by concave sections 133a, 133b and 133c, i.e., a trilobular-shaped cross section. The concave sections 133a, 133b and 133c allow the bag 12 to furl into a tighter configuration due to the geometry of the concave sections 133a, 133b and 133c. Moreover, each concave edge 134a, 134b and 134c of the bag brim 130 facilitates gripping the bag 12 during furling allowing a tighter and more secure furl. The concave edges 134a, 134b and 134c of the bag brim 130 may include a high friction surface to facilitate gripping the bag 12 when furling.

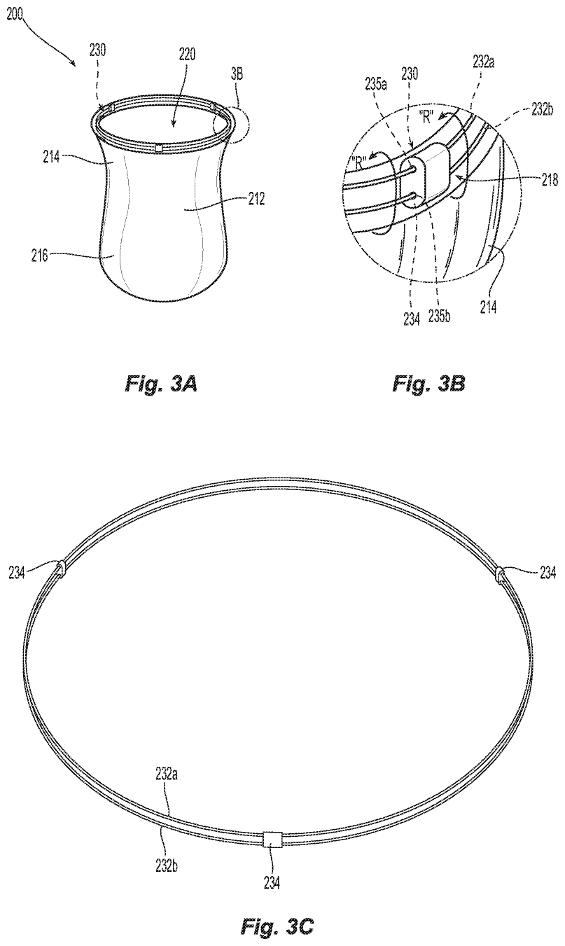

FIGS. 3A-3B show another embodiment of a tissue specimen retrieval bag assembly 200 for use with containing and supporting tissue specimens “T” within a surgical cavity. More particularly, bag assembly 200 includes a bag brim 230 having a bag 212 that depends therefrom for containing a tissue specimen “T”. Bag brim 230 is disposed within a cuff 218 defined in a proximal end 214 of the bag 212. Bag brim 230 includes a pair of wires 232a, 232b that form the bag brim frame. A series of connectors or spacers 234 are spaced about the bag brim 230 and connect the two wires 232a, 232b together. More particularly, each connector 234 includes a pair of throughholes 235a, 235b defined therein that are each configured to receive a respective wire 232a, 232b of the bag brim 230. The number of connectors 234 depends on the type of wire 232a, 232b and the size of the bag brim 230. More connectors 234 will provide more consistent spacing along the bag brim 230.

In use and much like the embodiments shown in FIGS. 2A and 2B, a distal end 2116 of the bag 212 is placed within an operating cavity and a tissue specimen “T” is positioned through opening 220 of bag 212. The geometry of the bag brim 230 facilitates furling/unfurling the bag 212 as needed and secures the bag 212 in a desired furled position. More particularly, the shape of the bag brim 230 facilitates furling the bag 212 and the brim 230 in the direction of rotation “R” over on itself which, in turn, allows the surgeon to position the tissue specimen “T” at a desired depth “L” (See for example, FIG. 5B) within the surgical cavity depending upon a particular purpose.

The wires 232a, 232b may be made of any know wire that is flexible such that the bag brim 230 is easily transitionable between a first collapsed configuration wherein the bag 212 is furled and a second expanded configuration which allows the bag 212 to be unfurled for receipt of a tissue specimen “T”, e.g., a shape memory alloy (SMA) such as nickel-titanium alloy commonly sold under the tradename Nitinol® or copper-aluminum-nickel. Bag 212 may include any suitable rollable material such as nylon, polyurethane, etc.

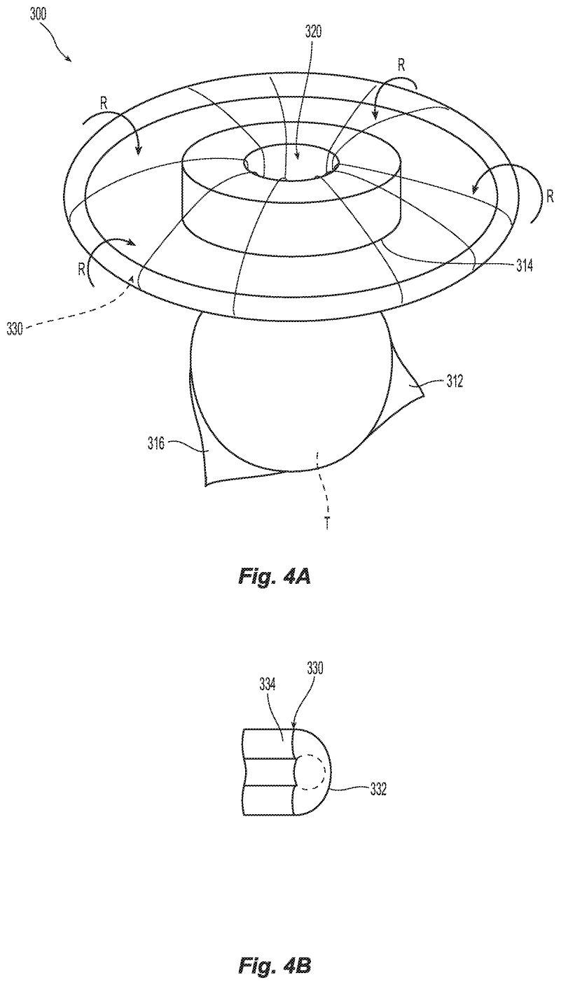

FIGS. 4-5C show another embodiment of a tissue specimen retrieval bag assembly 300 for use with containing and supporting tissue specimens “T” within a surgical cavity. More particularly, bag assembly 300 includes a bag brim 330 having a bag 312 that depends therefrom for containing a tissue specimen “T”. Bag brim 330 is disposed within a cuff (not shown) defined in a proximal end 314 of the bag 312. Bag brim 330 is generally D-shaped and includes an arcuate side 332 and a generally flat side 334. Much like the embodiments shown above, the generally D-shaped bag brim 330 allows the bag 312 to roll or furl around itself which, in turn, allows a surgeon to tent (i.e., position) the tissue specimen “T” closer to the proximal end of the operating cavity as needed for dissection.

More particularly, and as best shown in FIGS. 5A-5C, the geometry of the sides 332, 334 of the bag brim 330 facilitate furling/unfurling the bag 312 as needed and securing the bag 312 in a desired furled position. As shown in FIG. 5A, the specimen “T” is place into the specimen bag 312 through opening 320. The weight of the specimen “T” causes the specimen “T” to fall to toward the distal end 316 of the bag 312. The proximal end 314 of the bag 312 and the circularly-shaped bag brim 330 maintain the proximal end 314 the bag 312 outside the operating cavity (See FIG. 5C). If the surgeon desires to bring the specimen “T” closer to the proximal end 314 of the bag 312, the surgeon furls the bag 312 around the bag brim 330 in the direction “R”. The D-shape of the bag brim 330 facilitates furling the bag 312 and the brim 330 over on itself which, in turn, allows the surgeon to position the tissue specimen “T” at a desired depth “L” within the surgical cavity depending upon a particular purpose (See FIG. 5B). The flat side 334 and/or the arcuate side 332 may include a high friction surface to facilitate gripping the bag 312 when furling.

From the foregoing and with reference to the various drawings, those skilled in the art will appreciate that certain modifications can be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A tissue specimen retrieval bag assembly, comprising:
a tissue specimen bag having an open proximal end and a closed distal end, the proximal end including a cuff defined therein and extending therearound; and
a flexible bag brim disposed within the cuff, the flexible bag brim transitionable between a first, collapsed configuration and a second, expanded configuration, the flexible bag brim including a cross section configured to facilitate both furling the tissue specimen bag onto itself around the bag brim when the bag brim is disposed in the second, expanded configuration and securing the tissue specimen bag in a desired furled configuration.

2. The tissue specimen retrieval bag assembly according to claim 1 wherein the cross section includes a D-shaped cross section.

3. The tissue specimen retrieval bag assembly according to claim 1 wherein the cross section includes a triangular cross section.

4. The tissue specimen retrieval bag assembly according to claim 3 wherein the triangular cross section includes three sides, each of the three sides covered with a high friction material to facilitate gripping the tissue specimen bag when furling.

5. The tissue specimen retrieval bag assembly according to claim 1 wherein the cross section includes a trilobular-shaped cross section.

6. The tissue specimen retrieval bag assembly according to claim 5 wherein the trilobular-shaped cross section includes three edges defining three concave sections therebetween, the edges configured to facilitate gripping the tissue specimen bag when furling.

7. The tissue specimen retrieval bag assembly according to claim 1 wherein the cross section includes a D-shaped cross section, the D-shaped cross section including a flat side and an arcuate side, at least one of the sides including a high friction material to facilitate gripping the tissue specimen bag when furling.

8. The tissue specimen retrieval bag assembly according to claim 1 wherein the tissue specimen bag is made from at least one of nylon or polyurethane.

9. A tissue specimen retrieval bag assembly, comprising:
a tissue specimen bag having an open proximal end and a closed distal end, the proximal end including a cuff defined therein and extending therearound; and
a flexible bag brim disposed within the cuff, the flexible bag brim including a pair of flexible wires separated by at least one connector, the pair of wires transitionable between a first, collapsed configuration and a second, expanded loop-like configuration, the connector configured to space the wires relative to one another along the length of the bag brim, the pair of wires and the at least one connector configured to facilitate both furling the tissue specimen bag onto itself around the bag brim when the bag brim is disposed in the second, expanded, loop-like configuration and securing the tissue specimen bag in a desired furled configuration.

10. The tissue specimen retrieval bag assembly according to claim 9 wherein the tissue specimen bag is made from at least one of nylon or polyurethane.

11. The tissue specimen retrieval bag assembly according to claim 9 wherein the pair of wires is made from a shape memory alloy.

12. The tissue specimen retrieval bag assembly according to claim 9 wherein the shape memory alloy includes at least one of nickel-titanium alloy or copper-aluminum-nickel.
